(19) United States
(12) Patent Application Publication
(10) Pub. No.: US 2009/0216299 A1
(43) Pub. Date: Aug. 27, 2009

(54) System for Low-Level Laser Radiation

(75) Inventor: Marcos Dantus, Okemos, MI (US)

Correspondence Address: HARNESS, DICKEY & PIERCE, P.L.C., P.O. BOX 828, BLOOMFIELD HILLS, MI, 48303, US

(73) Assignee: Borad of Trustees of Michigan State University, East Lansing, MI (US)

(21) Appl. No.: 11/918,030

(22) PCT Filed: Apr. 5, 2006
(86) PCT No.: PCT/US2006/012793

§ 371 Date: Jan. 9, 2009

Related U.S. Application Data
(60) Provisional application No. 60/668,844, filed on Apr. 6, 2005.

(51) Int. Cl. A61N 5/06; G02B 6/26

(52) U.S. Cl. 607/89; 385/27

(57) ABSTRACT

A low-level laser therapy radiation system is provided. In a further aspect of the present invention, the system includes a laser source and an optical device. In another aspect of the present invention, a low-level laser therapy chamber is employed. The chamber produces an even distribution of laser radiation to a surface of a human body.

### CROSS REFERENCE TO RELATED APPLICATIONS

This application claims priority to U.S. Provisional Application Ser. No. 60/668,844, filed Apr. 6, 2005, which is incorporated by reference herein.

### BACKGROUND OF THE INVENTION

The present invention relates generally to a low-level laser therapy radiation device and more specifically to a systemic delivery system for low-level laser therapy radiation.

The use of low-level laser therapy radiation to provide musculoskeletal pain relief, promote cosmetic rejuvenation, promote accelerated healing of open and closed wounds as well as numerous other benefits has long been known. However, devices for use in such treatments have generally been designed as hand-held units that deliver radiation to areas smaller than two inches in diameter, which require skilled users to deliver treatment to particular locations of a human body. Furthermore, since conventional devices require an operator to physically aim and administer treatment, treatment results vary from patient to patient. On the other hand, devices that produce outputs at higher levels require special laser goggles by both a patient and an administering user. Moreover, conventional devices use 10 or less 0.5 mW infrared laser diodes and these may not produce optimal radiation levels to treat different regions of the human body.

### SUMMARY OF THE INVENTION

In accordance with the present invention, a low-level laser therapy radiation system is provided. In further aspect of the present invention, the system includes a laser source and an optical device. In another aspect of the present invention, a low-level laser therapy chamber is employed. The chamber produces an even distribution of laser radiation to a surface of a human body. A further aspect of the present invention includes a cavity therapeutic system. In another embodiment of the present invention, a low-level laser eye radiation device is employed. In an additional aspect of the present invention, a low-level laser healing device is provided. A further aspect of the present invention includes a hand-held device. In another aspect of the present invention, a low-level radiation therapy chair is employed.

The present invention provides a uniquely designed device for use in low-level laser therapy (LLLT) radiation. For example, the coherent and directionalized light allows for about 3-5 mm light penetration into the patient's skin. The present invention advantageously provides a systemic delivery of LLLT to achieve the maximal healing effects of LLLT in the most convenient methods of delivery to a total surface of a human body, internal parts of a human eye and/or other designated treatment areas of the human body. Additionally, the present invention minimizes accidental damage to the eyes of the operator or patient without an aid or use of protective goggles. Furthermore, the present invention is flexible and convenient for treatments to localized regions of the body without a need for an operator to apply a point source for a specific period of time in order to deliver optimal and consistent results from treatment. Moreover, one aspect of the present invention is based on a laser diode generating at least 100 mW in conjunction with diffractive optics to deliver LLLT to large areas of the human body such that a targeted area receives optimal laser radiation levels to promote and stimulate healing processes with cells. Additionally, the present invention promotes healing of a human eye by maximizing laser radiation exposure and by evenly applying laser radiation to an entire retina of the human eye. Furthermore, advantages and features of the present invention will become apparent from the following description and appended claims, taken in conjunction with the accompanying drawings.

### BRIEF DESCRIPTION OF THE DRAWINGS

The present invention will become more fully understood from the detailed description and the accompanying drawings, wherein:

FIG. 1 is a perspective view of a chamber according to a first preferred embodiment of the present invention system;

FIG. 2 is a diagrammatic side view of the chamber according to the present invention system of FIG. 1;

FIG. 3 is a cross-sectional view showing an optical device according to the present invention system of FIG. 1;

FIG. 4 is a diagrammatic end view of an alternative chamber embodiment of the present invention system;

FIG. 5 is a diagrammatic top view of a plurality of laser diode bars used within the alternative chamber embodiment of FIG. 4 of the present invention system;

FIG. 6 is a diagrammatic side view of a diode bar used in the alternative chamber embodiment of FIG. 4 of the present invention system;

FIG. 7 is a diagrammatic view of a low-level laser eye device according to a second preferred embodiment of the present invention system;

FIG. 8 is a diagrammatic view of a first alternative low-level laser eye device according to another embodiment of the present invention system;

FIG. 9 is a diagrammatic view of a second alternative low-level laser eye device according to another embodiment of the present invention system;

FIG. 10 is a diagrammatic view of a low-level radiation device according to another alternate embodiment of the present invention system;

FIG. 11 is a diagrammatic view of a hand-held device according to a fourth preferred embodiment of the present invention system;

FIG. 12 is a diagrammatic view of a low-level radiation chair according to another embodiment of the present invention system;

FIG. 13 is a perspective view of a portable patch device according to a third preferred embodiment of the present invention;

FIG. 14 is a diagrammatic front view of portable low-level radiation devices according to another alternate embodiment of the present invention system; and

FIG. 15 is a diagrammatic top view of the portable low-level radiation devices of FIG. 14 of the present invention system.

### DETAILED DESCRIPTION OF THE PREFERRED EMBODIMENTS

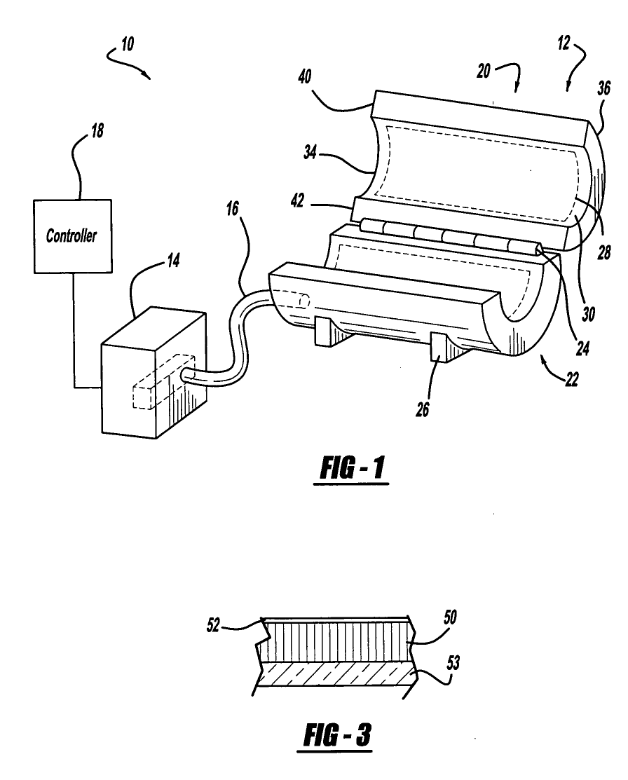

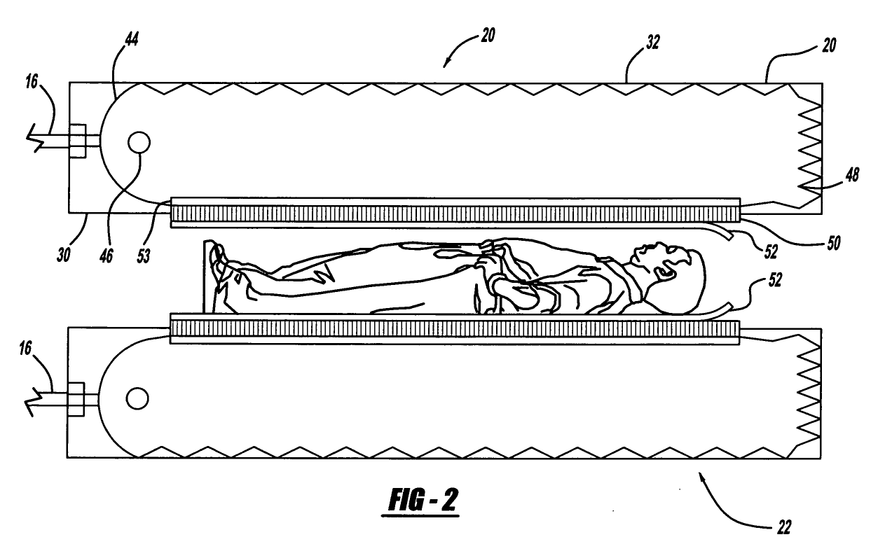

Referring to FIGS. 1-3, a first preferred embodiment of the present invention shows a low-level laser radiation system 10. System 10 includes a chamber, cavity or enclosure 12, a laser power supply 14, a transmission medium 16 and a programmable computer controller 18.

Chamber 12 is coupled to laser power supply 14 by transmission medium 16, such as a fiber optic cable. Chamber 12 operably receives at least one infrared laser beam or a coherent infrared laser light. Additionally, chamber 12 guides and evenly spreads the infrared laser beam throughout chamber 12 and onto a surface of a human body to stimulate and promote healing of cells within the surface. Chamber 12 is sufficiently large enough to enclose an adult body. Chamber 12 includes an upper concave optical unit 20, a lower concave optical unit 22, a set or plurality of pivotal devices 24 and a support structure 26. Laser power supply 14 includes a power supply having at least one infrared laser diode. Laser power supply 14 excites the laser diode and emits at least one infrared laser beam within a wavelength of 600 to 1100 nm. Additionally, laser power supply 14 outputs a total average power ranging from 3.6 to 36 kW. Transmission medium 16 transmits the infrared laser beam to upper concave optical unit 20 and lower concave optical unit 22 of chamber 12. Upper concave optical unit 20 and lower concave optical unit 22 include a length greater than or about 6 ft 5 inches (approximately 1.928 m) and a width greater than or about 3 ft (approximately 0.91 m). Upper concave optical unit 20 includes an upper optical assembly, arrangement or configurement 28, an inner concave surface 30, an outer convex surface 32, a pair of oppositely disposed ends 34 and 36, and a set of longitudinally edges 40 and 42.

Upper optical assembly 28 includes a concave structure 44, a dispersion sphere or device 46 and an undulated structure or member 48. Dispersion sphere 46 is a ball of aluminum having a microcrystalline and rough zinc oxide coating that scatters light. The sphere is about 1 cm diameter. Upper optical assembly 28 includes an upper length and an upper width. Upon receipt of the infrared laser beam, concave structure 44 having a white inner coating, reflects most, if not all or 100%, of the infrared laser beam into dispersion sphere 46. Dispersion sphere 46 distributes the infrared laser beam evenly throughout a length and a width of undulated structure 48, where the length is less than or about the upper length of upper optical assembly 28. Additionally, the width of undulated structure 48 is less than or about the upper width of upper optical assembly 28. Undulated structure 48 comprises a concave wave-like transparent material, such as polymeric material or glass. Additionally, undulated structure 48 reflects a dispersed laser beam, using internal reflection properties, onto inner concave surface 30.

Inner concave surface 30 includes an optical device, such as a waveguide window 50 having a diffusing element. Additionally, waveguide window 50 is less than or about the length and the width of upper concave optical unit 20 and manufactured into a concave form. Waveguide window 50, a diffractive optical device, includes a polymeric material, a glass material, or a ulexite material. The polymeric material may be an acrylic material, such as a Lumisty™ film material manufactured by Decorative Films, LLC as Model MFX-1515. The film acts as a fiber optic face plate but at a fraction of the cost. Furthermore, the film includes generally parallel partitions or wall-like internal features spaced from each other a distance closer than the film's thickness. These partitions channel or guide the light passing between the partitions in a directionalized manner. Two or more layers of the film are placed upon each other at 900 orientations to each other, however, different angles or numbers of layers can alternately be employed. Additionally, waveguide window 50 comprises a fused fiber optical device, such as a fiber optic face plate or alternately a fiber optic taper which may be manufactured by SCHOTT North American, Inc. The fiber optic taper, a polymeric device, is manufactured to comprise a thickness of about 1 mm. Waveguide window 50 magnifies and evenly spreads the laser beam along a predetermined path to reach a surface of the human body. The predetermined path is defined by the physical structure and/or material properties of the waveguide window 50. The diffusing element, a polymeric material, minimizes the laser beam from focusing within a specific region of the surface and a human eye by scattering and propagating the laser beam throughout chamber 12. The diffusing element ensures that the evenly spread laser beam delivers optimal benefits while the surface of the human body is less than 10 mm away from the diffusing element. Additionally, the diffusing element scatters and propagates the laser beam into non-coherent light as the evenly spread laser beam travels more than approximately 10 mm from the diffusing element.

The diffusing material may optionally include a directional pattern to reflect the evenly spread laser beam away from and minimize damage to the human eye. The directional pattern may include a horizontal pattern or a combination pattern having a horizontal and a vertical pattern. The horizontal pattern is constructed across the width of waveguide window 50. The vertical pattern is constructed across the length of waveguide window 50. The diffusing element operably transmits light with a narrow incidence cone and spreads the coherent infrared light into a non-coherent light. The cone is preferably in the range of about 1-20 degrees of normal incidence from the optical waveguide film, and more preferably, approximately 15 degrees. Additionally, waveguide window 50 may be manufactured sufficiently thick to minimize cracking and maintain durability such that waveguide window 50 may withstand constant contact by the human body and numerous hours of low-level laser therapy. On the other hand, a transparent structure or member 53, such as a polycarbonate material, is optionally utilized to support waveguide window 50, where transparent structure 53 is sufficiently thick to minimize waveguide window 50 from cracking and maintain durability, such that waveguide window 50 and transparent structure 53 may withstand constant contact by the human body and numerous hours of low-level laser therapy. Transparent structure 53 is less than or about a length and a width of waveguide window 50.

Lower concave optical unit 22 is a mirror image of upper concave optical unit 20 comprising all the same features and functions thereof. Upper concave optical unit 20 and lower concave optical unit 22 radiate the evenly spread laser beam throughout an area ranging less than or about 3,600,000 mm². Upper concave optical unit 20 and lower concave optical unit 22 disperse the evenly spread laser beam with an optical output ranging from 1 to 10 mW/mm².

Support structure 26 bears a weight of upper concave optical unit 20 and lower concave optical unit 22. Optionally, support structure 26 may contain other optical, electronic and/or mechanical devices needed by chamber 12. Pivotal devices 24, such as a hinge device, allow movement and guiding of upper concave optical unit 20 to lower concave optical unit 22 between a first or open position and a second or closed position. Additionally, pivotal devices 24 provide an acute angular movement of upper concave optical unit 20 relative to lower concave optical unit 22.

Controller 18 calibrates laser power supply 14 to emit the infrared laser beam for a specific amount of time. Additionally, controller 18 determines an amount of laser beam output delivered by system 10 to a surface or treating area of a human body. Controller 18 also determines a type of laser beam, a pulse or continuous wave, delivered to the surface of the human body. For optimal benefits, a duration for the pulse wave ranges from 10 to 100 nanoseconds. Additionally, controller 18 calibrates a pulse repetition rate delivered by laser power supply 14 to allow enough time between pulses for thermal relaxation to occur to the surface of the human body.

Optionally, chamber 12 may include a first or upper tanning unit and a second or lower tanning unit within upper concave optical unit 20 and lower concave optical unit 22, respectfully. The first tanning unit and the second tanning unit are less than or about the width and the length of upper concave optical unit 20 and lower concave optical unit 22. The first tanning unit and the second tanning unit dispenses ultraviolet light onto the surface of the human body in order to cause a darkening of a surface color or to produce a brown or tawny surface color of the human body.

Optionally, a thin transparent film 52 may be temporally coupled to waveguide window 50. Additionally, transparent film 52, a disposable material, is removed and applied to upper concave optical unit 20 and lower concave optical unit 22 after each use by the human body. By removing and reapplying transparent film 52, waveguide window 50 remains sanitary and minimizes the spreading of germs and/or viruses by the human body.

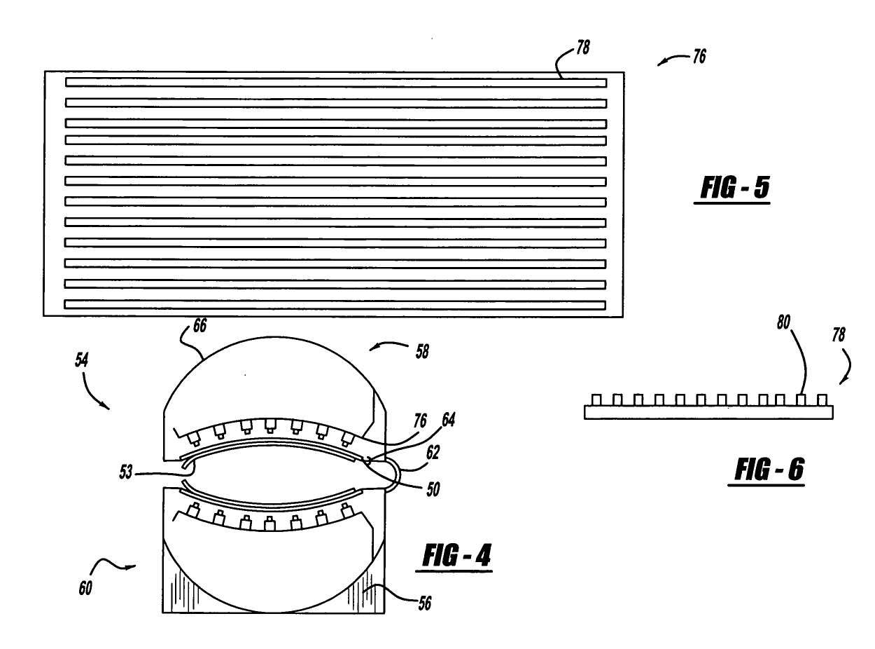

An alternative embodiment of the present invention comprises a cavity, housing, capsule or chamber therapeutic device 54, as shown in FIGS. 4-6. Cavity therapeutic device 54 evenly distributes an infrared laser beam or a coherent infrared light onto a surface of a human body to simulate and promote healing of cells within the surface. Cavity therapeutic device 54 includes a controller, a support structure 56, an upper concave laser assembly 58, a lower concave laser assembly 60 and a pair of pivotal devices 62. Additionally, cavity therapeutic device 54 is sufficiently large enough to enclose an adult human body.

Upper concave laser assembly 58 includes an inner concave surface 64, an outer convex surface 66, a pair of oppositely disposed ends, a set of longitudinally elongated edges, and a laser device or source 76. Additionally, upper concave laser assembly 58 includes an upper length and an upper width. Inner concave surface 64 includes a waveguide window 50 having a diffusing element. Waveguide window 50 having the diffusing element comprises all the same features and functions as those stated in the first preferred embodiment of the present invention for waveguide window 50 except as otherwise stated herein. A length of waveguide window 50 is less than or about the upper length. A width of waveguide window 50 is less than or about the upper width. Optionally, a transparent structure 53 is coupled to waveguide window 50 and supports waveguide window 50. Transparent structure 53 comprises all the same features and functions as those stated in the first embodiment of the present invention except as otherwise stated herein.

Laser device 76 is coupled to inner concave surface 64. Laser device 76 includes a plurality of laser bars 78. Each laser bar 78 is evenly spaced along the upper length such that the laser bars 78 cover less than or about the upper width. Additionally, each laser bar 78 comprises a specific number of laser diodes 80 less than or about a length of each diode bar 78. The specific distribution of laser diodes 80 is evenly spaced along each diode bar 78. Each laser diode 80 comprises an infrared diode. Additionally, each laser diode 80 comprises a diode greater than 100 mW, such as a 200 mW diode. Laser device 76 emits at least one infrared laser beam or a coherent infrared light received by inner concave surface 64. Upper concave laser assembly 58 and lower concave laser assembly 60 are mirror images thereof, containing identical features and functions.

Optionally, cavity therapeutic device 54 may include a first or upper tanning unit and a second or lower tanning unit within upper concave laser assembly 58 and lower concave laser assembly 60, respectively. The first tanning unit and the second tanning unit are less than or about a width and a length of upper concave laser assembly 58 and lower concave laser assembly 60. The first tanning unit and the second tanning unit dispenses ultraviolet light onto the surface of the human body in order to cause a darkening or a brown or tawny color on the surface of the human body.

Support structure 56 bears a weight of upper concave laser assembly 58 and lower concave laser assembly 60. Additionally, support structure 56 incorporates the controller and may contain other optical, electronic and/or mechanical devices needed by the cavity therapeutic device. Pivotal devices 62, such as a hinge device, allows for movement and guiding of upper concave laser assembly 58 to lower concave laser assembly 60 for a plurality of positions, such as between a first or open position and a second or closed position. Pivotal devices 62 provide an acute angular movement of upper concave laser assembly 58 relative to lower concave laser assembly 60.

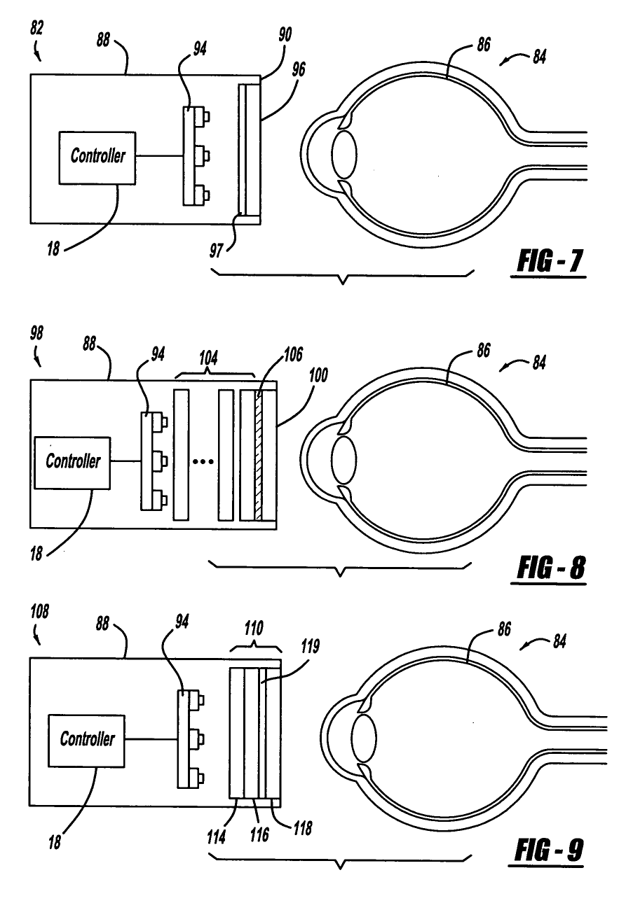

FIG. 7 refers to a second preferred embodiment of the present invention providing a low-level laser radiation device or unit 82 to distribute low-level laser radiation to at least one human eye 84 in order to stimulate processes in eye cells to promote healing within the human eye 84. Laser radiation device 82 exposes a retina 86 of the human eye 84 to at least one infrared laser beam or coherent infrared light. The infrared laser beam is evenly spread and scattered over retina 86 such that a laser beam does not focus and/or concentrate on a specific region of retina 86, which causes eye damage. Laser radiation device 82 includes a housing 88 having an eye surface 90, a laser source 94, and a programmable controller 18. Controller 18 is coupled to laser source 94. Laser source 94 is coupled to eye surface 90. Laser source 94, such as at least one laser 200 mW diode, emits at least one infrared laser beam onto eye surface 90. After receiving the infrared laser beam, eye surface 90, a waveguide device, window or member 96, expands the infrared laser beam up to 100 times while maintaining optimum dimensionality of the plurality of laser beams. Additionally, an optimal power dispersed by waveguide window 96 ranges from 0.05 to 0.5 W to the human eye 84. Waveguide window 96 treats an area of less than or about 100 mm². Waveguide window 96, a diffractive optical device, includes a polymeric material, a glass material, or a ulexite material. The polymeric material may be an acrylic material, such as a Lumisty™ material. Additionally, waveguide window 96 may preferably comprise a fiber optic face plate or alternately a fused fiber optical taper device. Additionally, waveguide window 96 spreads a reflected laser beam along a predetermined path onto a surface of the human body. The predetermined path is defined by a physical structure and/or material properties of waveguide window 96. Waveguide window 96 has a diffusing element to ensure that the infrared laser beam is evenly spread over retina 86 of human eye 84. Optimal benefits are achieved when human eye 84 is located within a range less than or about 10 mm from the diffusing element. Additionally, the diffusing element produces a non-coherent light as the evenly spread laser beam travels farther than approximately 10 mm from the diffusing element or waveguide window 96. Additionally, waveguide window 96 may be formed sufficiently thick to minimize cracking and maintain durability such that waveguide window 96 may withstand constant contact by the human body and numerous hours of low-level laser therapy. On the other hand, a transparent structure or member 97, a polycarbonate material, is optionally utilized to support waveguide window 96. Transparent structure 97 is sufficiently thick to minimize waveguide window 96 from cracking maintain durability, such that waveguide window 96 and transparent structure 97 may withstand constant contact by the human body and numerous hours of low-level laser therapy. Transparent structure 97 is less than or about a length and a width of waveguide window 96.

Housing 88 is coupled to waveguide window 96, laser source 94 and controller 18. Additionally, housing 88 is stationary and includes adjustable components or elements to adapt to different eye levels and pupil sizes for different patients. Additionally, laser radiation device 82 may include a second eye surface such that laser radiation device 82 may deliver low-level laser radiation to a left human eye and a right human eye. Laser radiation device 82 may further include electronics or mechanics to deliver low-level laser radiation to the left human eye or the right human eye separately and simultaneously.

A first alternative of the low-level radiation device of the present invention is an eye radiation device or unit 98, as shown in FIG. 8. Eye radiation device 98 includes a laser source 94, a controller 18, an eye optical window 100, and a housing 88. Controller 18 is coupled to laser source 94. Laser source 94 is coupled to eye optical window 100. Laser source 94 comprises all the same features and functions as stated in the second preferred embodiment for laser source 94 of the present invention. Eye optical window 100 includes a plurality of diffractive optical devices 104, such as a plurality of beam splitters. Laser source 94 emits at least one infrared laser beam or a coherent laser source onto diffractive optical devices 104. Diffractive optical devices 104 are placed in a first layer and a second layer such that the infrared laser beam expands up to 100 times and evenly spreads the laser beam while maintaining optimum dimensionality of the laser beam. Diffractive optical devices 104 are stacked against one another such that the laser beam is split evenly into a multiple of laser beams by the first layer of diffractive optical device 104. Next, the multiple of laser beams are further split into a greater multiple of laser beams by the second layer of diffractive optical devices 104. Diffractive optical devices 104 evenly spreads the laser beam such that an average power output by the evenly spread laser beams range from 0.1 to 1 W onto a treating area of about 100 mm². Eye optical window 100 includes an optical surface having a waveguide window. The waveguide window comprises all the same functions and features as described in the second preferred embodiment for its waveguide window except as otherwise stated herein. Optionally, a transparent structure may be utilized to support the waveguide window.

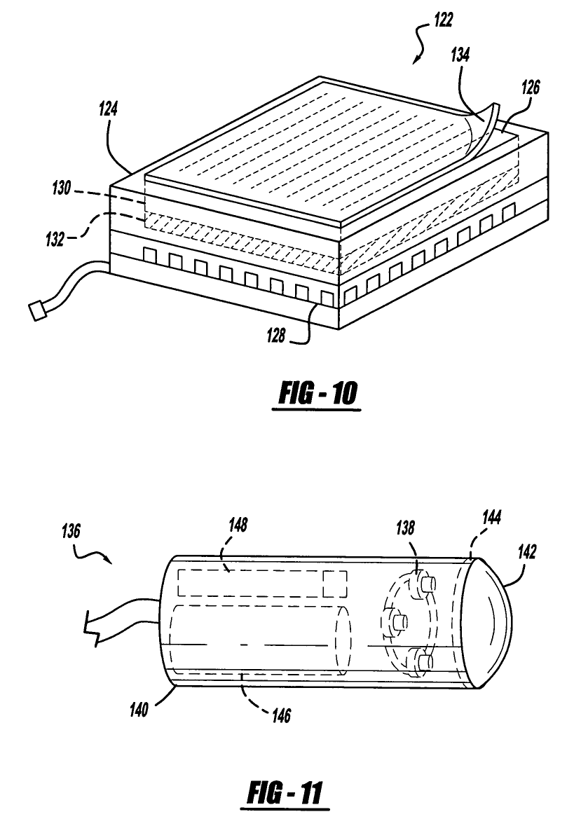

As shown in FIG. 9, a second alternative of the low-level laser radiation device of the present invention system is an eye laser device 108. Eye laser device 108 includes an eye optical unit 110, a laser source 94, a housing 88 and controller 18. Eye optical unit 110, having a waveguide device, window or unit 96, distributes evenly spread laser beams to a total area of a retina 86 of a human eye 84. Eye optical unit 110 includes a plurality of optical devices comprising a first layer 114, a second layer 116, and a third layer 118. Laser source 94 is coupled to first layer 114. First layer 114 is in turn coupled to second layer 116 and second layer 116 is in turn coupled to third layer 118. Upon receipt of the laser beam, first layer 114, which is a scattering optical unit, randomly scatters and splits the laser beam into a multiple laser beams. Next, second layer 116, comprising a transparent optical unit such as a total internal reflection device, receives and reflects the multiple beams into third layer 118. Third layer 118 is a waveguide unit comprising all the same functions and features as the waveguide window in the second preferred embodiment of the present invention except as otherwise stated herein. Optionally, a transparent member may be utilized to support waveguide unit 118. Waveguide unit 118 produces an average power output ranging from 0.05 to 0.5 W to a treating area of about 100 mm².

As shown in FIG. 10, an alternate embodiment of the present invention is a low-level laser therapy device 122. Device 122, a pad-like device, is sufficiently thin and flexible to contour to a treating surface and underlying body members of a human body. A thickness of device 122 may be less than or about 0.125 inches. Device 122 operably receives at least one infrared laser beam or a coherent infrared light and evenly spreads the infrared laser beam to stimulate and promote healing of cells within the treating surface. Additionally, device 122 is sufficiently large enough to cover an upper body portion or a lower body portion of the human body. On the other hand, device 122 may be sufficiently small enough to cover small portions of the human body. Device 122 generally covers a surface area of less than or about 10,000 mm². Device 122 includes a housing 124 having a surface plate 126, programmable microprocessor-based controller 18, and a laser source 128. Controller 18 is coupled to laser source 128 and laser source 128 is coupled to surface plate 126. Housing 124 encloses controller 18 and laser source 128. Laser source 128 comprises a plurality of evenly spaced 200 mW diodes. The diodes are evenly spaced along a length and a width of surface plate 126. Laser source 128 emits at least one infrared laser beam to surface plate 126. Surf ace plate 126, an optical device, includes a waveguide device, window or unit 130 having a diffusing element. Waveguide device 130 is a diffractive optical device including a polymeric material, a glass material, or a ulexite material. The polymeric material may be an acrylic material, such as a Lumisty™ material. Additionally, waveguide device 130 comprises a plurality of fused optical fibers that guides and spreads the laser beam along a predetermined path and onto the surface of the human body. The plurality of fused optical fibers are preferably fiber optic face plates or alternately fiber optic tapers. The predetermined path is defined by the physical structure and/or material properties of waveguide device 130. Waveguide device 130 ensures that an evenly spread laser beam delivers optimal benefits as the surface of the human body is less than or about 10 mm from surface plate 126. The diffusing element, such as a Lumisty™ material or other polymeric material, minimizes the evenly spread laser beam from focusing within a specific region of the surface of the human body. Additionally, the diffusing element scatters and propagates the laser beam into a non-coherent light as the evenly spread laser beam travels more than approximately 10 mm from surface plate 126 to minimize damage to a human eye. Optionally, a plurality of optical devices may be used to magnify, increase or further spread the evenly spread laser beam to cover a larger area on the surface of the human body. Additionally, waveguide device 130 may be formed sufficiently thick to minimize cracking and maintain durability such that waveguide device 130 may withstand constant contact by the human body and numerous hours of low-level laser therapy. On the other hand, a transparent structure or member 132, a polycarbonate material, is optionally utilized to support waveguide device 130. Transparent structure 132 is sufficiently thick to minimize waveguide device 130 and transparent structure 132 from cracking and maintain durability, such that waveguide device 130 may withstand constant contact by the human body and numerous hours of low-level laser therapy. Transparent structure 132 is less than or about a length and a width of waveguide device 130. Additionally, a transparent film 134, a disposable material, is removed and applied after each use by the human body. By disposing and applying transparent film 134 after device 122 is used by the human body, waveguide device 130 minimizes the spreading of germs and/or viruses by the human body for sanitary purposes.

A third preferred embodiment of the low-level laser radiation system 180 is shown in FIG. 13. System 180 is generally in the form of a luminous pad or bandage, and preferably includes a flexible, light emitting polymer (LEP) also known as a polymeric light emitting diode (PLED), or alternately an organic light emitting diode (OLED) 182 having a thickness of about 1-2 mm and a periphery of about 25 mm². The PLED can be obtained from Cambridge Display Technologies and contains an emissive material applied on a substrate in a manner like ink jet printing without a vacuum. The OLED can be obtained from Eastman-Kodak Co. in a “small-molecule” type mode by vacuum deposition. OLED 182 generates infrared light having a wavelength of about 600-800 nm, and about 0.1-1 mW per mm² intensity. An exemplary OLED is disclosed in U.S. Patent Publication No. 2005/0230678 entitled “Organic Electronic Device Comprising Conductive Members and Processes for Forming and Using the Organic Electronic Device” to Cao et al., which is incorporated by reference herein. An antistick, perforated and polymeric film, or a gauze-like sheet 184 is adhered to a generally flat face of PLED or OLED facing the patient's skin. An optional polymeric housing for a battery is adhered, stapled or otherwise attached adjacent to an edge of PLED or OLED 182. A 1.5 volt or other button cell, watch-style battery 181 is connected to a conductive film 183 by wires or stamped metal contacts 190. An adhesively attachable switch contact 192 causes battery 186 to energize conductive film 183 which energizes PLED or OLED 182 when the switch completes the circuit. The battery, switch and wires/stampings define at least part of an electrical circuit. It should be appreciated that alternate electric circuits, including a programmable microprocessor-based controller, a voltage regulator, and/or solid state electronics, may be employed to activate PLED or OLED. Accordingly the system is adhesively taped or otherwise held against a person's or animal's skin with gauze film 184 over the wound or area to be treated. It is expected that the system can be used for about one or two days, and thereafter discarded.

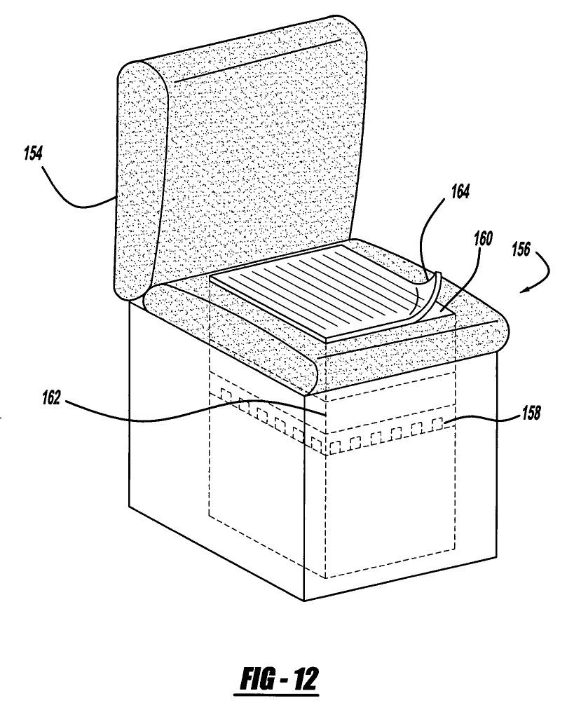

FIG. 11 refers to a fourth preferred embodiment of the present invention comprising a low-level laser hand-held or wand device 136. Hand-held device 136 treats a surface area of a human body ranging from 100 to 1,000 mm². Additionally, a maximum power output by hand-held device 136 ranges from 1 to 10 mW/mm². Hand-held device 136 is sufficiently light weight and portable such that device 136 may be held in a hand of an operator for periods of time while administering laser treatment to localized areas of the patient's body. Hand-held device 136 comprises a programmable controller, a laser source 138, and a housing 140 having an optical surface or device 142. The controller is coupled to laser source 138, laser source 138 is coupled to optical surface 142, and the controller and laser source 138 are affixed to housing 140. Optical surface 142, a diffractive optical device, is molded into a three-dimensional dome shape. Additionally, optical surface 142, here a waveguide device or window, has a diffusing element including a polymeric, glass material or ulexite material. The polymeric material may be an acrylic, such as a Lumisty™ material. Additionally, waveguide device 142 may comprise a fiber optic face plate or fused fiber optic taper. Optical surface 142 guides the laser beam along a predetermined path and spreads the laser beam evenly onto a surface of the human body. The predetermined path is defined by a physical structure and/or material properties of waveguide device 142. Waveguide device 142 ensures that the evenly spread laser beam delivers optimal benefits when the surface of the human body is less than or about 10 mm from waveguide device 142. The diffusing element, a polymeric material, minimizes the evenly spread laser beam from focusing within a specific region of the surface and entering into a human eye by scattering and propagating the laser beam into a non-coherent light as the evenly spread laser beam travels more than approximately 10 mm from optical surface 142. Additionally, waveguide device 142 may be formed sufficiently thick to minimize cracking and maintain durability such that waveguide device 142 and transparent structure 144 may withstand constant contact by the human body and numerous hours of low-level laser therapy. On the other hand, a polycarbonate transparent structure or member 144 is optionally utilized to support waveguide device 142, where transparent structure 144 is sufficiently thick to minimize waveguide device 142 from cracking and maintain durability, such that waveguide device 142 may withstand constant contact by the human body and numerous hours of low-level laser therapy. Transparent structure 144 is less than or about a length and a width of waveguide device 142.

Additionally, a clear or transparent gel substance may be used for lubricating the hand-held unit for improved motion and contact with the surface of the human body. Optionally, hand-held device 136 may include an ultrasonic device 146. Ultrasonic device 146 emits high frequency pulses into the human body in order to treat targeted cells, tumors and lesions. Additionally, hand-held device 136 may include a rechargeable battery 148, as a power source. Rechargeable battery 148 is coupled to the controller and located within housing 140. Optionally, hand-held device 136 may incorporate a stand or structure unit such that hand-held device 136 may be free standing and supported by the stand to deliver the laser radiation at a particular location. The stand eliminates a need for an operator to hold hand-held device 136 thereby delivering optimal and consistent results.

FIG. 12 depicts a fifth preferred embodiment of the present invention system referring to a low-level laser radiation chair 152. Chair 152 includes a programmable controller, a back structure 154, a base structure 156, and a laser source 158. Back structure 154 is coupled to base structure 156. Base structure 156 is coupled to the controller and laser source 158, where the controller is coupled to laser source 158. Laser source 158 includes a plurality of laser diodes, such as 200 mW diodes, where the laser diodes are evenly spaced over a length and a width of base structure 156, and emit at least infrared one laser beam or a coherent infrared light. Additionally, base structure 156 includes an optical surface 160 and at least two side surfaces to raise base structure 156 a distance from a ground, such that an adult may sit comfortably upon base structure 156 while receiving low-level radiation therapy. The optical surface 160 is a waveguide window, unit or device having a diffusing element which emits a plurality of diffused laser beams or a coherent infrared light directly to a treating area of a human body. Waveguide window 160, a diffractive optical device, is sufficiently large enough to allow low-level laser treatment for a pelvic area related problem, such as hemorrhoids, scrotal related problems, prostate and vaginal inflammations, while the patient is sitting. Waveguide window 160, such as a polymeric Luminsty™ material, a glass material, or a ulexite material, guides and evenly spreads the laser beam along a predetermined path onto a surface of the human body. Additionally, waveguide window 160 may comprise a fiber optic face plate or a plurality of fused optical fibers, such as a fiber optic taper. The predetermined path is defined by the physical structure and/or material properties of waveguide window 160. Waveguide window 160 ensures that the evenly spread laser beam delivers optimal benefits when the surface of the human body is less than or about 30 mm from optical surface 160. The diffusing element, a polymeric material, minimizes the evenly spread laser beam from focusing within a specific region of the treating surface by scattering and propagating the laser beam into a non-coherent light as the evenly spread laser beam travels more than approximately 30 mm from waveguide window 160. Additionally, waveguide window 160 may be formed sufficiently thick to minimize cracking and maintain durability such that waveguide window 160 may withstand constant contact by the human body and numerous hours of low-level laser therapy. On the other hand, a polymeric, transparent structure or member 162 is optionally utilized to support waveguide window 160. Transparent structure 162 is sufficiently thick to minimize waveguide window 160 and transparent structure 162 from cracking and maintain durability, such that waveguide window 160 may withstand constant contact by the human body and numerous hours of low-level laser therapy. Transparent structure 162 is less than or about a length and a width of waveguide window 160.

Additionally, chair 152 is capable of dispensing laser beams with a power ranging from 0.5 to 5 W. Optionally, a disposable, thin transparent film 164 is temporally coupled to waveguide window 160. By removing and applying transparent film 164, waveguide window 160 remains sanitary and minimizes the spreading of germs and/or viruses from the human body.

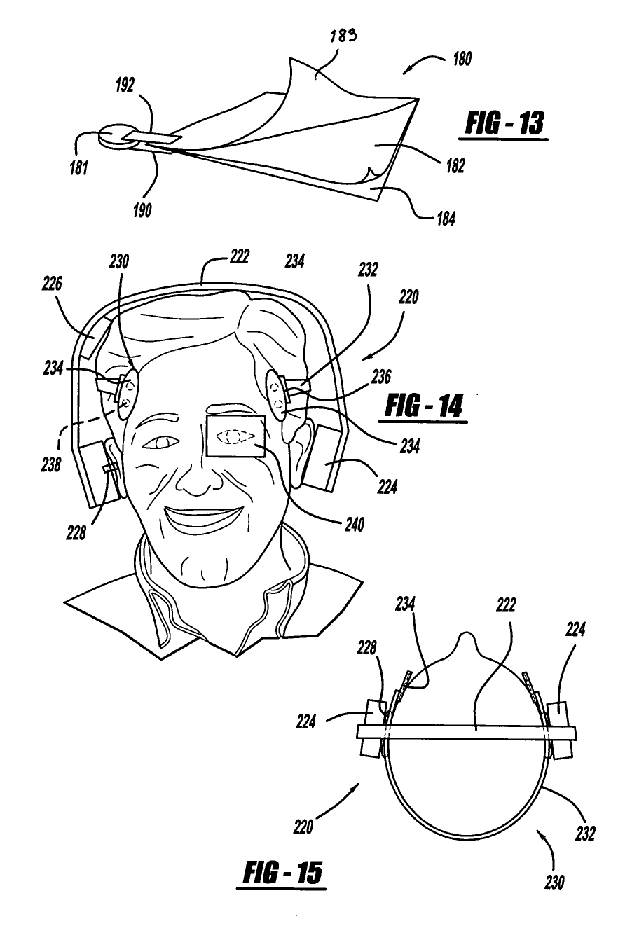

FIGS. 14 and 15 illustrate various additional alternate embodiments of the present invention low-level laser radiation system. An ear device 220 employs a flexible and adjustable, head-mounted band 222, upon each end of which is mounted an apparatus 224 like that shown in FIG. 7, 8 or 9. A soft foam cushion surrounds the housing of each apparatus 224 but with a central hole through which a light emitting diode 228 extends. A distal end of each diode 228 is located and aimed in the patient's ear canal to treat ear problems. A battery 226 is attached to band 222 and connected to both diodes 228, the associated programmable, microprocessor-based electronics and the associated electrical circuit.

A temple device 230 has a flexible head-mounted band 232, portions of which are removable worn on or above the patient's ears. Housings or pads 234 are attached to forward ends of band 232 and contain multiple light emitting diodes 238 directed in a radial manner, generally perpendicular to and aimed at the patient's temple or alternately, forehead. A battery 236, an associated controller and an electrical circuit are mounted to band 232. This device is used to treat headaches or the like.

Furthermore, an eye patch device 240 can be taped over the patient's eye to treat eye or eyelid ailments. Device 240 can be of the type shown in FIG. 10 or 13.

While various aspects of the present invention have been disclosed, it should be appreciated that variations may be made without departing from the scope of the present invention. For example, an optical device may include multiple layers of diffractive optical elements. Additionally, many embodiments of the present invention have stated power ranges for laser outputs, area coverage ranges by a particular low-level laser device, distance ranges detailing a prescribed distance for maximum benefit while operating a specific embodiment. However, it should be appreciated that while the stated ranges are for optimal performance, the specific device may operate outside of those stated ranges. Furthermore, while many of the embodiments herein include a laser source within an embodiment, the laser source may be external and remote from a housing or enclosure and at least one laser beam may be transmitted using a laser power supply. Such an embodiment may include a transmitting material from the laser power supply coupled to the portable housing. Additionally, an electrical or mechanical device and/or arrangement may be used instead of an optical device to achieve the same or similar goals of the present invention. Furthermore, chamber 12 and cavity 54 may employ a patient bed such that a human body may lie down on the bed during a treatment session. The patient bed may include a lining and a cushion. The lining of the patient bed may be made of a polymeric, leather or textile material. The polymeric material may be an acrylic material. Additionally, a thin transparent film may be included in all embodiments for sanitary purposes minimizing the spreading of germs and/or viruses from a human body. Moreover, some advantages of the present invention may not be realized in the alternative embodiments. Furthermore, various materials have been disclosed in an exemplary fashion, but other materials may of course be employed, although some of the advantages of the present invention may not be realized. It is intended by the following claims to cover these and any other departures from the disclosed embodiments, which fall within the true spirit of the invention.

1. A low-level laser system comprising:
a chamber operably receiving a coherent light; and
at least one optical device coupled to the chamber and receiving the coherent light;
the at least one optical device guiding, directionalizing along one or more substantially predetermined paths, and evenly spreading the coherent light throughout the chamber and directed to at least a head and a majority of a body of a patient inside the chamber.

2. The low-level laser system of claim 1 further comprising a transparent member to support the at least one optical device, wherein the transparent member is sufficiently thick to withstand weight of the patient.

3. The low-level laser system of claim 1 wherein the at least one optical device is sufficiently thick to withstand weight of the patient, and the at least one optical device includes a film that obscures light transmission therethrough at a majority of angles but not others.

4. The low-level laser system of claim 1 wherein the at least one optical device further comprises a diffusing element operably scattering and propagating the coherent light into a non-coherent light as the coherent light travels more than or about 10 mm from the diffusing element, and wherein the coherent light is coherent infrared light.

5. The low-level laser system of claim 1 further comprising:
at least one dispersion device receiving the coherent light, the at least one dispersion device reflecting and dispersing the coherent light;
an undulated structure coupled to the dispersion device and receiving a dispersed coherent light, the undulated structure spreading the dispersed coherent light substantially over a length and a width of the undulated structure, wherein the undulated structure guides and reflects the dispersed coherent light onto the at least one optical device.

6. The low-level laser system of claim 1 wherein the at least one optical device comprises a directional patterned material transmitting an evenly spread coherent light guided by substantially parallel partitions in a film, the patritions being closer together than the thickness of the film.

7. The low-level laser system of claim 1 wherein the at least one optical device comprises a waveguide.

8. The low-level laser system of claim 1 wherein the at least one optical device comprises a diffusing element operably transmitting light within a narrow incidence cone and spreading the coherent infrared light into a non-coherent light.

9. The low-level laser system of claim 1 wherein the optical device delivers an optimal dosage of coherent infrared light to the patient while the patient is less than or about 10 mm from the optical device.

10. The low-level laser system of claim 1 wherein the chamber is sufficiently large enough to enclose the patient which is a human, further comprising a laser source emitting the infrared light having an average power intensity of about 1 to 36 kW.
a laser source emitting the infrared light having an average power intensity of about 1 to 36 kW.

11. The low-level laser system of claim 1 wherein the coherent light comprises a pulsed wave.

12. The low-level laser system of claim 1 wherein the coherent light comprises a continuous wave.

13.-19. (canceled)

20. A low-level radiation device comprising:
a laser source producing at least one infrared laser beam; and
a waveguide coupled to the laser source and evenly spreading the at least one laser beam onto a retina area of an eye.

21.-31. (canceled)

32. A low-level laser therapy apparatus comprising:
an infrared laser source operably emitting a coherent and substantially infrared light; and
a member including an optical device coupled to the laser source, the optical device comprises a plurality of light guiding elements, and at least a section of the member including the optical device supporting a portion of a human body;
wherein the optical device receives the coherent and substantially infrared light, directionalizes the coherent and substantially infrared light using the light guiding elements in one or more substantially predetermined directions toward a treatment region of the human body for a distance of about 1 cm from the optical device, and thereafter the directionality of the emitted light is diffused after about 1 cm from the optical device which minimizes human eye safety concerns.

34. The low-level laser therapy apparatus of claim 32 further comprising a transparent member coupled to the optical device and supporting the optical window, wherein the transparent member supports a majority of the human body and a human head in a substantially horizontal orientation.

35. The low-level laser therapy apparatus of claim 33 wherein the member includes a chair.

36. (canceled)

37. The low-level laser therapy apparatus of claim 32 wherein the optical device comprises a fiber optic face plate.

38. The low-level laser therapy apparatus of claim 32 wherein the optical device comprises a diffusive polymeric film which scatters and propagates the coherent infrared light into a non-coherent light.

39. The low-level laser therapy apparatus of claim 32 further comprising a sphere dispersing the infrared light.

40. The low-level laser therapy apparatus of claim 32 further comprising an upper unit movably coupled to a lower unit by a hinge, the member being attached to at least one of the units.

41.-54. (canceled)

55. A low-level radiation therapy chair comprising:
at least one optical device operably receiving a coherent infrared light, the at least one optical device initially directionalizing the light and thereafter diffusing the light, wherein the at least one optical device evenly spreads the coherent infrared light source onto a human pelvic area; and
a base structure having a surface coupled to the at least one optical device, wherein the at least one optical device administers substantially evenly spread coherent infrared light to the human pelvic area while a human body sits upon the surface.

56. The low-level radiation therapy chair of claim 55 wherein the at least one optical device comprises a waveguide film.

57. (canceled)

58. The low-level radiation therapy chair of claim 55 wherein the at least one optical device comprises a directional patterned material reflecting the even spread of coherent light.

59.-78. (canceled)

79. A method of applying low-level laser therapy to a patient, the method comprising:
(a) placing at least a portion of the patient into a chamber;
(b) emitting low-level laser light into the chamber; and
(c) substantially evenly distributing the laser light to the patient in the chamber with a waveguide.

80.-82. (canceled)
